(19) United States
(12) Patent Application Publication
(10) Pub. No.: US 2019/0038400 A1
(43) Pub. Date: Feb. 7, 2019

(54) Corneal Transplant Systems, Methods, and Apparatuses

(71) Applicant: Sandeep Samudre, Chesapeake, VA (US)

(72) Inventor: Sandeep Samudre, Chesapeake, VA (US)

(21) Appl. No.: 16/075,857

(22) PCT Filed: May 17, 2017
(86) PCT No.: PCT/US17/33192

§ 371 Date: Aug. 6, 2018

Related U.S. Application Data
(60) Provisional application No. 62/338,108, filed on May 18, 2016.

(51) Int. Cl. A61F 2/14

(52) U.S. Cl. CPC A61F 2/148; A61F 2/1691; A61F 2/142

(57) ABSTRACT

A transplant tissue storage, transport, and/or placement apparatus, including at least some of a transplant device extending from a distal portion to a proximal portion; a tissue support element formed in the distal portion, the tissue support element having an upper surface having a tissue support element configured to support a central portion of transplant tissue, and a transplant tissue securing mechanism with opposing first securing pad and the second securing pad configured for clasping opposing peripheral portions of transplant tissue in a draped configuration; and a lockable arrangement formed in the proximal portion, the lockable arrangement configured to release a secured transplant tissue from the mounting surface, upon actuation.

### CROSS-REFERENCE TO RELATED APPLICATIONS

This patent application claims the benefit of U.S. Patent Application Ser. No. 62/338,108, filed May 18, 2016, the entire disclosure of which is incorporated herein by reference.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### NOTICE OF COPYRIGHTED MATERIAL

The disclosure of this patent document contains material that is subject to copyright protection. The copyright owner has no objection to the reproduction by anyone of the patent document or the patent disclosure, as it appears in the Patent and Trademark Office patent file or records, but otherwise reserves all copyright rights whatsoever. Unless otherwise noted, all trademarks and service marks identified herein are owned by the applicant.

### BACKGROUND OF THE PRESENT DISCLOSURE

### 1. Field of the Present Disclosure

The present disclosure relates generally to the field of corneal transplant surgical systems, methods, and apparatuses. More specifically, the presently disclosed corneal transplant systems, methods, and devices allow for improved corneal transplant.

### 2. Description of Related Art

Over the last 10 years, corneal transplantation (keratoplasty) has been evolving from a technique of transplanting all layers of the cornea (deep penetrating keratoplasty) in the central field of vision to replacing only the diseased layer of the cornea. One technique is called Descemet's Membrane Endothelial Keratoplasty (DMEK). Utilizing this technique, only the Descemet's membrane and endothelium, the innermost layers of the cornea, are grafted onto the recipient's cornea. This technique has many benefits, including faster healing time and minimal refractive error, accrued by eliminating additional scar transplant tissue formation. A potential downfall for DMEK is the primary graft failures that occur in a third of cases because of improper graft excision and placement during surgery. Currently, the allografts are excised from whole corneas at the bedside prior to surgeries. This is done under conditions that often comprise integrity of the delicate transplant tissue.

A compromised allograft usually goes undetected in the operating room environment because current procedures bypass quality control measures employed in the transplant tissue-processing laboratory. At this time, appropriate instrumentation to handle this delicate DMEK graft has not been developed.

DMEK allows for transplanting only the diseased layer of the cornea. Transplantation of the posterior layer of the cornea, the endothelium, accounts for over 40% of all corneal transplant surgeries and this number is growing. During the DMEK procedure, the donor cornea endothelium is dissected. To prepare the recipient cornea to receive this transplant tissue, only the endothelium is removed, leaving the full native thickness of the recipient's cornea intact. Although the thickness of the graft is only approximately 100 μm, any non-symmetrical cuts in the corneal graft and/or improper placement introduces a refractive error into the recipient's vision.

Any discussion of documents, acts, materials, devices, articles, or the like, which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE PRESENT DISCLOSURE

If the donor transplant tissue were able to be processed in a transplant tissue-processing lab rather than the bedside, it would be possible to use a single human donor cornea for more than one patient or procedure. Since 1905, the entire thickness of the cornea was used in a transplant. Through use of the corneal transplant devices and systems of the present disclosure, a transplant tissue bank may prepare only the required transplant tissue. The anterior portion of the cornea may then be used for patients with other anterior corneal maladies.

Current DMEK surgeries do not use a proper transplant device. Currently, when the transplant tissue is introduced into the recipient's eye, the transplant tissue floats freely in the anterior chamber of the recipient's eye, making it difficult to properly position the transplant tissue. Transportation of the transplant tissue is also problematic, as there is no current device that can safely hold and transport the delicate transplant tissue. The corneal transplant devices and systems of the present disclosure can allow storage, transportation, and accurate placement of transplant tissues.

To overcome these and various other shortcomings in the current art, the exemplary, nonlimiting embodiments of the transplant tissue storage, transport, and/or placement systems may optionally include at least some of a container vessel, a support tray, and a transplant device for precise placement of transplant tissues during corneal transplantation. The transplant device may take the form of a stylus that receives pre-cut transplant tissue in the distal end with a release mechanism on the proximal end. Located in the distal end of the transplant device are transplant tissue securing mechanisms that grip and secure the transplant tissue once placed. These transplant tissue securing mechanisms may be configured to receive transplant tissues of various sizes. Along with the articulation, either fixed or removable, and transplant tissue securing mechanisms, the distal end may be curved to mimic concavity of the eye and ensure that transplant tissue is placed precisely and retained at the transplant site in the recipient's eye.

In certain exemplary, nonlimiting embodiments, during transplantation, the distal end containing the transplant tissue may be inserted into the eye with the proximal end used to maneuver the transplant tissue. The proximal end of the transplant device may be configured to accommodate the angle of approach during transplantation. The surgeon grips the shaft in the proximal end of the transplant device and has the ability to release a locking mechanism when the desired placement of the transplant tissue in the eye has been achieved. Once unlocked, the transplant tissue is released in the distal end. The support tray may be configured to retain the transplant device during transport. The support tray may or may not be coupled to the transplant device. The container vessel may be configured to receive either the transplant device, support tray, or both. The system may be configured to assist corneal surgeons perform transplants in a rapid, precise and safe manner.

In certain exemplary, nonlimiting embodiments, the system for precise corneal transplantation may include a container vessel, transplant device, and a support tray. The transplant device receives pre-cut transplant tissue and places it precisely in the recipient's eye. Located in the distal end of the transplant device are mechanisms that grip and secure the transplant tissue. The distal end may be curved to mimic concavity of the eye to ensure precise placement of the transplant tissue. During transplantation, the distal end containing the transplant tissue is inserted into the eye with the proximal end used to maneuver the transplant tissue. The transplant tissue may be released through actuating a locking mechanism. The support tray may receive the transplant device for transport and either one or both could be transported by placement in the container vessel.

In certain exemplary, nonlimiting embodiments, the transplant devices and methods described herein are optionally configured for use in the storage, transport, and/or placement of a variety of transplant tissues of various sizes. For example, the transplant device (also referred to herein as “apparatus”) can include a container vessel and/or a support tray. The container vessel may optionally be configured to accept the transplant device as well as make it possible to image the transplant tissue in vitro. The support tray may optionally be configured to accept the transplant device for restraint during transport. The transplant device may optionally remain disposed within the container vessel until the transplant procedure. The transplant device therefore allows for the transplant tissue to be prepared at a transplant tissue bank and mounted on the transplant device instead of at the bedside prior to transplant surgery, which represents a valuable improvement over contemporary techniques. The use of this device also introduces a key element of quality control introducing consistency in transplant tissue quality.

In traditional endothelial transplantation procedures, the donor transplant tissue is processed at the bedside. The excised transplant tissue is then folded in half and inserted into the anterior chamber of the recipient's eye through a small incision. During the insertion, this delicate transplant tissue is usually grasped with forceps and once in the anterior chamber, the transplant tissue is difficult to manipulate in place because of its natural folding pattern. It is important to reduce manipulation events to preserve endothelial cell density throughout the entire transplant procedure. Several devices that are currently developed to reduce manipulation during transplant tissue insertion, or transplant tissue injector devices, only address the transplant tissue insertion into the anterior chamber of the eye and do not adequately address the precise placement of the transplant tissue at the transplant site. With the transplant devices and methods described herein, a transplant tissue may optionally be implanted into a recipient's eye with minimal manipulation as it is inserted into the eye and placed at the appropriate implantation site.

In certain exemplary, nonlimiting embodiments, the transplant device of the present disclosure may optionally be a single-use, disposable device that facilitates transport and precise placement of quality pre-cut cornea allografts. The transplant device is introduced into the recipient's anterior chamber through a small incision through the cornea or sclera as part of an endothelial keratoplasty procedure and its variations.

In certain exemplary, nonlimiting embodiments, the transplant device of the present disclosure may optionally be used by, for example, a transplant tissue bank or an eye bank to prepare and distribute various endothelial transplant tissue preparations at the specific request of the surgeon. In this usage, the specific endothelial transplant tissue is mounted on to a device, placed in a sterile container vessel containing preservative media and then packaged and shipped to the surgical center.

Thus, in certain exemplary, nonlimiting embodiments, the transplant device of the present disclosure may optionally be used to transport pre-cut transplant tissue, used in endothelial keratoplasty procedure and its variants, such as Posterior Lamellar Keratoplasty (PLK) or Deep Lamellar Endothelial Keratoplasty (DLEK), Descemet Stripping (Automated) Endothelial Keratoplasty (DS(A)EK), Descemet Membrane Endothelial Keratoplasty (DMEK). The pre-cut transplant tissue may optionally be mounted on to a transplant tissue securing mechanism of the transplant device prior to delivery. The transplant device as described herein may optionally also be used to transport and place synthetic lenses, such as those used for cataract surgeries, as well as shunts, such as those used in glaucoma surgeries.

In certain exemplary, nonlimiting embodiments, the transplant device may optionally comprise of a transplant device that may optionally insert and place corneal allografts into the recipient's eye. This may optionally reduce the overall manipulation of the transplant tissue and the overall surgical time. Thus, the transplant device mentioned herein may optionally make the surgical procedure easier and convenient option for the surgeon as it eliminates several steps during the transplant process, namely the need for bedside preparation of the donor transplant tissue, and easier placement of the allograft once introduced into the recipient's eye.

As described herein, in an endothelial keratoplasty procedure, the distal portion of the transplant device containing the transplant tissue may optionally, for example, be placed into the anterior chamber of the recipient's eye through a corneal or scleral incision. The incision is the entry point for the transplant device and the size may optionally be between 4 mm to 9 mm. Once inside the anterior chamber, the transplant device may optionally be guided to the appropriate placement site by the surgeon. Once the desired placement site has been identified, the surgeon may optionally gently guide the transplant tissue into position using the distal portion of the transplant device, and actuate a release mechanism of the transplant device's proximal portion to release the transplant tissue. In certain exemplary, nonlimiting embodiments, the release mechanism may take the form of an articulating arm on the transplant device's proximal portion.

The transplant device may optionally be loaded with the corneal allograft in an environment such as a transplant tissue bank or eye bank with appropriate sterile processing facilities. All (or less than all) portions of the transplant device may optionally be provided in a sterile and disposable format to reduce the chance for contamination and infection. The transplant tissue-processing technician follows proper sterile technique to pre-cut and mount the corneal allograft on to the transplant device. The transplant tissue along with the transplant device may optionally then be placed in the container vessel, containing a storage media, such as Optisol™ (Bausch and Lomb, Rochester, N.Y.). In certain exemplary, nonlimiting embodiments, a guide may optionally be included with the transplant device to stabilize and reduce mechanical stress on the corneal allograft during transport. The process described above can save substantial time and effort on the surgeon as well as increase the quality of the donor transplant tissue thereby increasing overall success of the surgical outcome.

The donor cornea transplant tissue may optionally be prepared on demand as per the surgeon's specifications. In certain exemplary, nonlimiting embodiments, transplant tissue may optionally be pre-cut, and preloaded in the transplant device in advance of a transplant procedure for up to 72 hours in an appropriate storage media. The storage media, in some cases may optionally preserve the endothelial cell counts for up to two weeks if stored at 4 degrees C.

The relative dimensions, the rounded distal head and articulating arm of the transplant device described herein may also facilitate placement and release of the transplant tissue with relative ease. The transplant tissue, when mounted on the transplant device, has restricted movement and therefore able to preserve the endothelial cell damage due to reduction in mechanical shock. This configuration is superior to the rolled transplant tissue configuration because it allows for less overall manipulation. Also, mounting the transplant tissue does not detract from the natural tendency of the transplant tissue to roll up, instead the transplant device mentioned herein controls the natural roll to give an advantage to the surgeon during placement without a negative impact on the endothelial surface. During transport, the secured transplant tissue and the transplant device are suspended in the container vessel, making no actual contact with the container vessel interior surfaces and reducing damage due to mechanical stress.

The distal end of the transplant device described herein, which receives the transplant tissue, is designed to hold the transplant tissue in place utilizing a transplant tissue securing mechanism. The spires, which grip the transplant tissue, are located on the first securing pad and the second securing pad and may optionally be of assorted heights, types and placement positions of these spires may optionally also vary. Also, some embodiments may optionally contain securing elements, facilitating easy dismount of the transplant tissue. This embodiment reduces the possibility of transplant tissue sticking to the distal end of the transplant device. The distal end of the transplant device described herein, in certain exemplary embodiments has a curved surface that mimics the natural curvature of the cornea. This curved surface is used to place the transplant tissue properly on the underside of the recipient's cornea. The curved may be smoothed so as to not stick to the transplant tissue.

In various exemplary, nonlimiting embodiments, the transplant tissue storage, transport, and/or placement apparatus comprises a transplant device extending from a distal portion to a proximal portion; a tissue support element formed in the distal portion, the tissue support element having an upper surface having a tissue support element configured to support a central portion of transplant tissue, and a transplant tissue securing mechanism with opposing first securing pad and the second securing pad configured for clasping opposing peripheral portions of transplant tissue in a draped configuration; and a lockable arrangement formed in the proximal portion, the lockable arrangement configured to release a secured transplant tissue from the mounting surface, upon actuation.

In certain exemplary, nonlimiting embodiments, the proximal portion comprises a first arm extending proximally from the mounting surface and a first opposing securing pad, and a second arm extending proximally from a second opposing securing pad, the first arm hingedly connected to the second arm.

In certain exemplary, nonlimiting embodiments, the transplant tissue securing mechanism comprises a first securing pad extending from the first arm, and a second securing pad extending from the second arm.

In certain exemplary, nonlimiting embodiments, the hinged connection is located on a side of the mounting surface opposite of the proximal portion.

In certain exemplary, nonlimiting embodiments, the transplant tissue storage, transport, and/or placement apparatus further comprises a transplant tissue container vessel with an interior chamber configured to retain a cornea allograft storage media and house at least one of the transplant device and a transplant tissue support tray.

In various exemplary, nonlimiting embodiments, the transplant tissue container vessel comprises a viewing lens configured to provide magnification of at least a portion of the inner chamber.

In various exemplary, nonlimiting embodiments, the container vessel receives the distal end of the transplant device and is fitted with a luer type lock.

In various exemplary, nonlimiting embodiments, the transplant device is configured to be removably inserted into the container vessel's inner chamber.

In various exemplary, nonlimiting embodiments, the transplant tissue support tray comprises a transplant tissue mounting and placement mount for removably securing the transplant device.

In certain exemplary, nonlimiting embodiments, the transplant tissue storage, transport, and/or placement apparatus further comprises a transplant tissue support tray with a transplant tissue mounting and placement mount for removably securing the transplant device.

In various exemplary, nonlimiting embodiments, the opposing first securing pad and the second securing pad comprise a first opposing securing pad and a second opposing securing pad, and at least one opposing securing pad comprises a plurality of protrusions on a transplant tissue contacting surface.

In certain exemplary, nonlimiting embodiments, the transplant tissue storage, transport, and/or placement apparatus further comprises a lip within the transplant tissue container vessel interior chamber configured to receive the transplant device and a lip on the proximal end to allow docking with the transplant device.

In various exemplary, nonlimiting embodiments, the mounting surface and the transplant tissue securing mechanism are configured to secure a transplant tissue to prevent movement and orient the transplant tissue with the endothelial side facing away from the mounting surface.

In various exemplary, nonlimiting embodiments, at least one securing pad comprises a plurality of protrusions on a transplant tissue contacting surface.

In various exemplary, nonlimiting embodiments, the first securing pad and the second securing pad each have a horseshoe-shaped transplant tissue contacting surface.

In various exemplary, nonlimiting embodiments, the upper surface of the mounting surface is smooth and dome-shaped to mimics the concavity of the transplant tissue in the draped configuration.

In various exemplary, nonlimiting embodiments, the opposing first securing pad and the second securing pad have a first side and a second side, the first side configured for clasping a first outer peripheral portion of the transplant tissue, and the second side configured for clasping a second outer peripheral portion of the transplant tissue.

In various exemplary, nonlimiting embodiments, the first outer peripheral portion of the transplant tissue is substantially opposite the second outer peripheral portion of the transplant tissue.

In various exemplary, nonlimiting embodiments, the transplant tissue storage, transport, and/or placement system comprises a transplant tissue container vessel with an interior chamber configured to retain a cornea allograft storage media; and a transplant device having a distal portion and a proximal portion, wherein: the distal portion having a tissue support element with an upper surface having a tissue support element configured to support a central portion of transplant tissue, and a transplant tissue securing mechanism with opposing first securing pad and the second securing pad configured for clasping opposing peripheral portions of transplant tissue in a draped configuration; and the proximal portion having an lockable arrangement configured to release a secured transplant tissue from the mounting surface upon actuation.

In certain exemplary, nonlimiting embodiments, the transplant tissue storage, transport, and/or placement apparatus further comprises a transplant tissue support tray with a transplant tissue mounting and placement mount for removably securing the transplant device, wherein the transplant tissue support tray comprises a distal groove and a medial groove, and the transplant devices includes a first mating region configured to removably mate with the distal groove and a second mating region configured to removably mate with the medial groove.

Accordingly, the presently disclosed systems, methods, and/or apparatuses separately and optionally provide corneal transplant devices and systems that allow storage of transplant tissues.

The presently disclosed systems, methods, and/or apparatuses separately and optionally provide corneal transplant devices and systems that allow transportation of transplant tissues.

The presently disclosed systems, methods, and/or apparatuses separately and optionally provide corneal transplant devices and systems that allow accurate placement of transplant tissues.

The presently disclosed systems, methods, and/or apparatuses separately and optionally provide corneal transplant devices and systems that allow improve surgical outcomes.

The presently disclosed systems, methods, and/or apparatuses separately and optionally provide corneal transplant devices that can be easily manipulated by a user.

These and other aspects, features, and advantages of the presently disclosed systems, methods, and/or apparatuses are described in or are apparent from the following detailed description of the exemplary, non-limiting embodiments of the presently disclosed systems, methods, and/or apparatuses and the accompanying figures. Other aspects and features of embodiments of the presently disclosed systems, methods, and/or apparatuses will become apparent to those of ordinary skill in the art upon reviewing the following description of specific, exemplary embodiments of the presently disclosed systems, methods, and/or apparatuses in concert with the figures. While features of the presently disclosed systems, methods, and/or apparatuses may be discussed relative to certain embodiments and figures, all embodiments of the presently disclosed systems, methods, and/or apparatuses may optionally include one or more of the features discussed herein. Further, while one or more embodiments may be discussed as having certain advantageous features, one or more of such features may also be used with the various embodiments of the systems, methods, and/or apparatuses discussed herein. In similar fashion, while exemplary embodiments may be discussed below as device, system, or method embodiments, it is to be understood that such exemplary embodiments may optionally be implemented in various devices, systems, and methods of the presently disclosed systems, methods, and/or apparatuses.

Any benefits, advantages, or solutions to problems that are described herein with regard to specific embodiments are not intended to be construed as a critical, required, or essential feature(s) or element(s) of the presently disclosed systems, methods, and/or apparatuses or the claims.

### BRIEF DESCRIPTION OF THE SEVERAL VIEWS OF THE DRAWINGS

As required, detailed exemplary embodiments of the presently disclosed systems, methods, and/or apparatuses are disclosed herein; however, it is to be understood that the disclosed embodiments are merely exemplary of the presently disclosed systems, methods, and/or apparatuses that may be embodied in various and alternative forms, within the scope of the presently disclosed systems, methods, and/or apparatuses. The figures are not necessarily to scale; some features may be exaggerated or minimized to illustrate details of particular components. Therefore, specific structural and functional details disclosed herein are not to be interpreted as limiting, but merely as a basis for the claims and as a representative basis for teaching one skilled in the art to employ the presently disclosed systems, methods, and/or apparatuses.

The exemplary embodiments of the presently disclosed systems, methods, and/or apparatuses will be described in detail, with reference to the following figures, wherein like reference numerals refer to like parts throughout the several views, and wherein:

FIG. 1A illustrates an exemplary embodiment of a corneal transplant device, according to the present disclosure, wherein the transplant device is in a substantially open configuration;

FIG. 1B illustrates an exemplary embodiment of a corneal transplant device, according to the present disclosure, wherein the transplant device is in a substantially closed configuration;

FIG. 2 illustrates a side view of an exemplary embodiment of a corneal transplant system, according to the present disclosure;

FIG. 3 illustrates a top perspective view of an exemplary embodiment of a corneal transplant system, according to the present disclosure;

FIG. 4 illustrates a bottom perspective view of an exemplary embodiment of a corneal transplant system, according to the present disclosure;

FIG. 5 illustrates a proximal end view of an exemplary embodiment of a corneal transplant device, according to the present disclosure;

FIG. 6 illustrates a distal end view of an exemplary embodiment of a corneal transplant device, according to the present disclosure;

FIG. 7 illustrates a front-side view of an exemplary embodiment of a screw cap, operable in connection with the corneal transplant device, according to the present disclosure;

FIG. 8 illustrates a side view of an exemplary embodiment of the first arm of the corneal transplant device, according to the present disclosure;

FIG. 9 illustrates a side view of an exemplary embodiment of a proximal end of the first arm of the corneal transplant device, of FIG. 8, according to the present disclosure and an exemplary embodiment of the first arm and second arm locking mechanism;

FIG. 10 illustrates a rear view of an exemplary embodiment of a second arm proximal end of an exemplary embodiment of the corneal transplant device, according to the present disclosure;

FIG. 11 illustrates a rear view of both first arm and second arms of an exemplary embodiment of the corneal transplant device, according to the present disclosure;

FIG. 12 illustrates a front view of an exemplary embodiment of a removable articulation of the corneal transplant device, according to the present disclosure;

FIG. 13 illustrates a side view of an exemplary embodiment of a removable articulation of the corneal transplant device, according to the present disclosure;

FIG. 14 illustrates a side view of an exemplary embodiment of a centrally located articulation of the corneal transplant device, according to the present disclosure;

FIG. 15 illustrates a side view of an exemplary embodiment of a proximally located articulation of the corneal transplant device, according to the present disclosure;

FIG. 16 illustrates a side view of an exemplary embodiment of transplant tissue gripping first securing pad and second securing pad of the corneal transplant device, according to the present disclosure;

FIG. 17 illustrates a top view of an exemplary embodiment of a transplant tissue gripping securing pad of the corneal transplant device with an exemplary embodiment of a spire pattern, according to the present disclosure;

FIG. 18A illustrates a side view of an exemplary embodiment of a lower transplant tissue gripping securing pad of the corneal transplant device with alternating spire types and heights, according to the present disclosure;

FIG. 18B illustrates a side view of an exemplary embodiment of first securing pad and the second securing pad depicted with asymmetric spire pattern, according to the present disclosure;

FIG. 19 illustrates a top-front perspective view of the securing element of the corneal transplant device, according to the present disclosure;

FIG. 20 illustrates a top-front perspective view of an exemplary embodiment of the securing element of the corneal transplant device, according to the present disclosure;

FIG. 21 illustrates a top-front perspective of the securing element placed in exemplary transplant tissue gripping first securing pad and second securing pad of the corneal transplant device, according to the present disclosure;

FIG. 22 illustrates a top view of an exemplary embodiment of an exemplary securing element of the corneal transplant device, according to the present disclosure;

FIG. 23 illustrates a side cross-sectional view of an exemplary embodiment of a container vessel of the corneal transplant device that allows top access to the transplant tissue cell, according to the present disclosure;

FIG. 24 illustrates a top view of an exemplary embodiment of a container vessel of the corneal transplant device that allows top access to the transplant tissue cell, according to the present disclosure;

FIG. 25 illustrates a side cross-sectional view of an exemplary embodiment of a container vessel of the corneal transplant device that allows side access to the transplant tissue cell, according to the present disclosure;

FIG. 26 illustrates a top view of an exemplary embodiment of a container vessel of the corneal transplant device that allows side access to the transplant tissue cell, according to the present disclosure;

FIG. 27 illustrates an exploded side view of an exemplary embodiment of a transplant tissue cell of an exemplary, nonlimiting embodiment of container vessel of the corneal transplant device, according to the present disclosure;

FIG. 28 illustrates a side view of an exemplary embodiment of the container vessel of the corneal transplant device, according to the present disclosure;

FIG. 29 illustrates a side, cross-sectional view of an exemplary embodiment of a transplant tissue container vessel of the corneal transplant device, according to the present disclosure;

FIG. 30 illustrates a cutaway front-side view of an exemplary embodiment of a coupling sleeve of the corneal transplant device, according to the present disclosure;

FIG. 31 illustrates a side cross-sectional view of an exemplary embodiment of a transplant tissue support tray that may be used in conjunction with the container vessel;

FIG. 32 illustrates a front perspective view of an exemplary embodiment of a transplant tissue support tray with orientation pins, according to the present disclosure;

FIG. 33 illustrates a front view of an exemplary embodiment of optional orientation pins of a transplant tissue support tray, according to the present disclosure;

FIG. 34 illustrates a front view of an exemplary embodiment of optional orientation pins of a transplant tissue support tray, according to the present disclosure; and

FIG. 35 illustrates a front perspective of an exemplary embodiment of optional orientation pins with top and bottom flanges of a transplant tissue support tray, according to the present disclosure.

### DETAILED DESCRIPTION OF EXEMPLARY EMBODIMENTS OF THE PRESENT DISCLOSURE

For simplicity and clarification, the design factors and operating principles of the corneal transplant systems, methods, and/or apparatuses are explained with reference to various exemplary embodiments of corneal transplant systems, methods, and/or apparatuses. The basic explanation of the design factors and operating principles of the corneal transplant device is applicable for the understanding, design, and operation of the corneal transplant device of the presently disclosed systems, methods, and/or apparatuses. It should be appreciated that the corneal transplant device can be adapted to many applications where corneal transplant device can be used.

As used herein, the word “may” is meant to convey a permissive sense (i.e., meaning “having the potential to”), rather than a mandatory sense (i.e., meaning “must”). Unless stated otherwise, terms such as “first” and “second” are used to arbitrarily distinguish between the exemplary embodiments and/or elements such terms describe. Thus, these terms are not necessarily intended to indicate temporal or other prioritization of such exemplary embodiments and/or elements.

The term “coupled”, as used herein, is defined as connected, although not necessarily directly, and not necessarily mechanically. The terms “a” and “an” are defined as one or more unless stated otherwise.

The term “exemplary” is used herein to mean “serving as an example, instance, or illustration”. Any embodiment described herein as “exemplary” is not necessarily to be construed as preferred or advantageous over other embodiments. Likewise, the term “embodiment” does not require that all embodiments of the invention include the discussed feature, advantage, or mode of operation.

Throughout this application, the terms “comprise” (and any form of comprise, such as “comprises” and “comprising”), “have” (and any form of have, such as “has” and “having”), “include”, (and any form of include, such as “includes” and “including”) and “contain” (and any form of contain, such as “contains” and “containing”) are used as open-ended linking verbs. It will be understood that these terms are meant to imply the inclusion of a stated element, integer, step, or group of elements, integers, or steps, but not the exclusion of any other element, integer, step, or group of elements, integers, or steps. As a result, a system, method, or apparatus that “comprises”, “has”, “includes”, or “contains” one or more elements possesses those one or more elements but is not limited to possessing only those one or more elements. Similarly, a method or process that “comprises”, “has”, “includes” or “contains” one or more operations possesses those one or more operations but is not limited to possessing only those one or more operations.

It should also be appreciated that the terms “transplant device”, “container vessel”, “allograft”, and “transplant tissue” are used for basic explanation and understanding of the operation of the systems, methods, and apparatuses of the presently disclosed systems, methods, and/or apparatuses. Therefore, the terms “transplant device”, “container vessel”, “allograft”, and “transplant tissue” are not to be construed as limiting the systems, methods, and apparatuses of the presently disclosed systems, methods, and/or apparatuses.

It should be noted that in this written description and the appended claims, the singular terms such as “graft”, “allograft”, and “Transplant tissue” are used interchangeably and include plural referents unless the context clearly dictates otherwise. Thus, for example, “graft” is intended to mean “Transplant tissue”. The term “Transplant tissue” refers to the processed endothelial layer that is being used for transplant purposes, unless otherwise noted. It should also be noted that the words “proximal” and “distal” refer to direction closer to and away from, respectively, an operator (e.g., surgeon, physician, nurse, technician, etc.) who would prepare the transplant tissue for loading and also the surgeon who would insert the transplant device into the anterior chamber of the patient's eye. Therefore, the end inserted inside a patient's eye would be the distal end of the transplant device, while the end outside a patient's body would be the proximal end of the transplant device.

For simplicity and clarification, the systems, methods, and/or apparatuses of the present disclosure are described as being used in conjunction with DMEK procedures and/or techniques. However, it should be appreciated that these are merely exemplary embodiments of the corneal transplant systems, methods, and/or apparatuses of the present disclosure and are not to be construed as limiting corneal transplant systems, methods, and/or apparatuses of the present disclosure. Thus, the corneal transplant systems, methods, and/or apparatuses of the present disclosure may be utilized in conjunction with any currently known or later developed corneal transplant procedures or techniques.

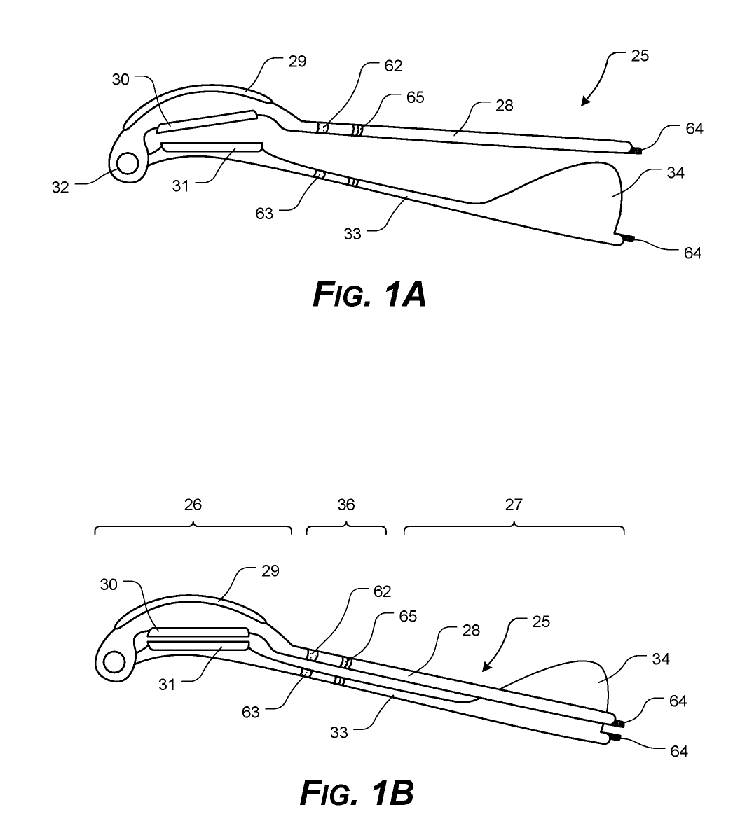

Turning now to the appended drawing figures, FIGS. 1A-35 illustrate certain elements and/or aspects of certain exemplary embodiments of the corneal transplant device 25, according to the presently disclosed systems, methods, and/or apparatuses.

As illustrated most clearly in FIGS. 1A-6, the transplant system 23 optionally comprises at least some of a container vessel 24, a transplant device 25, and a support tray 61. It should be appreciated that some or all of the container vessel 24, the transplant device 25, and/or the support tray 61 may optionally be included as the transplant system 23.

In various exemplary, non-limiting embodiments, the container vessel 24 may optionally comprise, for example, a vial, bag, blister pack, or other suitable container defining an interior chamber or cavity sized and configured to contain a transplant storage medium (e.g., Optisol™) and the transplant device 25. For example, the container vessel 24 may optionally be configured to contain a transplant storage medium and the transplant device 25. The container vessel 24 may optionally contain one or more restraint mechanisms, such as, for example, a support tray 61 to lock or support the transplant device 25 within the container vessel 24.

The transplant device 25 extends generally from a proximal portion 27, through a central portion 36, to a distal portion 26. The proximal portion 27 is generally configured to be grasped by the user and guide the transplant tissue 80 to a desired transplant location (i.e., an anterior chamber of an eye). In various exemplary embodiments, at least a portion of the proximal portion 27 and/or the central portion 36 provide an angle to the transplant device 25 for ease of access into the recipient's eye, during a transplant procedure.

The distal portion 26 includes a tissue support element 29 configured to receive and at least partially support the transplant tissue 80. In various exemplary embodiments, the tissue support element 29 is positioned atop a portion of the first arm 28, proximate the distal portion 26. At least a portion of the surface of the tissue support element 29 is formed so as to mimic the curvature of an exemplary cornea and forms a mounting surface (or at least part of a mounting surface).

The tissue support element 29 may optionally also be configured to place the transplant tissue 80 in the desired transplant location as described herein. If the tissue support element 29 mimics or follows the natural contour of the human cornea, the tissue support element 29 can be utilized to ensure accurate placement of the transplant tissue 80 in the recipient's eye.

The transplant device 25 comprises a first arm 28 and a second arm 33. The first arm 28 and second arm 33 are pivotably attached or coupled to one another, within the distal portion 26, via a pivot element 32. In this manner, the first arm 28 and the second arm 33 are able to pivot, via the pivot element 32, relative to one another, between a substantially open position (as illustrated in FIG. 1A) and a substantially closed position (as illustrated in FIG. 1B). In various exemplary embodiments, the pivot element 32 may optionally be fixed or removable. In certain exemplary, nonlimiting embodiments, the distal portion 26 of the transplant device 25 may optionally comprise a mechanism that receives and locks the transplant tissue 80 for storage, transport, and/or placement.

In various exemplary embodiments, the proximal portion 27 may optionally also include a release element 34. In various exemplary embodiments, the release element 34 comprises a “fin-like” projection extending from either the first arm 28 or the second arm 33 (as illustrated). If included, the release element 34 is positioned so as to ease the manipulation of the transplant device 25 from the closed position to the open position. Thus, facilitating the release of secured transplant tissue 80 from the tissue support element 29.

In certain exemplary embodiments, the first arm 28 and the second arm 33 may optionally be detachable from one another at points 62 and 63 in the first arm 28 and second arm 33, respectively.

The distal portion 26 of the transplant device 25 further comprises a transplant tissue securing mechanism that receives and secures at least a portion of the transplant tissue 80 to the tissue support element 29, for storage, transport, and/or placement. In various exemplary, nonlimiting embodiments, the transplant tissue securing mechanism may optionally resemble a “jaw” like structure that receives at least edge portions of the transplant tissue 80. In various exemplary embodiments, the transplant tissue securing mechanism comprises a first securing pad 30 and a second securing pad 31.

The first securing pad 30 extends from at least a portion of the first arm 28 and the second securing pad 31 extends from at least a portion of the second arm 33. The first securing pad 30 and second securing pad 31 are positioned proximate one another, such that surface portions of the first securing pad 30 and the second securing pad 31 are positioned adjacent one another, when the transplant device 25 is in the closed position. A distance between adjacent surface portions of the first securing pad 30 and the second securing pad 31, when the transplant device 25 is in the closed position, is a design choice based upon the desired amount of pressure to be exerted upon portions of the transplant tissue 80 to secure at least a portion of the transplant tissue 80 to the tissue support element 29.

In various exemplary embodiments, adjacent surface portions of the first securing pad 30 and the second securing pad 31 include surface features or preparations to ensure that the first securing pad 30 and second securing pad 31 are able to appropriately grasp and secure transplant tissue 80, when the transplant device 25 is in the closed position.

In various exemplary embodiments, various components of the transplant device 25 are substantially rigid and are formed of stainless steel. Alternate materials of construction of the various components of the transplant device 25 may include one or more of the following: steel, aluminum, titanium, polytetrafluoroethylene, and/or other metals, as well as various alloys and composites thereof, glass-hardened polymers, polymeric composites, polymer or fiber reinforced metals, carbon fiber or glass fiber composites, continuous fibers in combination with thermoset and thermoplastic resins, chopped glass or carbon fibers used for injection molding compounds, laminate glass or carbon fiber, epoxy laminates, woven glass fiber laminates, impregnate fibers, polyester resins, epoxy resins, phenolic resins, polyimide resins, cyanate resins, high-strength plastics, nylon, glass, or polymer fiber reinforced plastics, thermoform and/or thermoset materials, and/or various combinations of the foregoing. Thus, it should be understood that the material or materials used to form the various components of the transplant device 25 is a design choice based on the desired appearance and functionality of the transplant device 25.

It should be appreciated that certain elements of the transplant device 25 may be formed as an integral unit. Alternatively, suitable materials can be used and sections or elements made independently and attached or coupled together, such as by adhesives, welding, screws, rivets, pins, or other fasteners, to form the various elements of the transplant device 25.

It should also be understood that the overall size and shape of the transplant device 25, and the various portions thereof, is a design choice based upon the desired functionality and/or appearance of the transplant device 25.

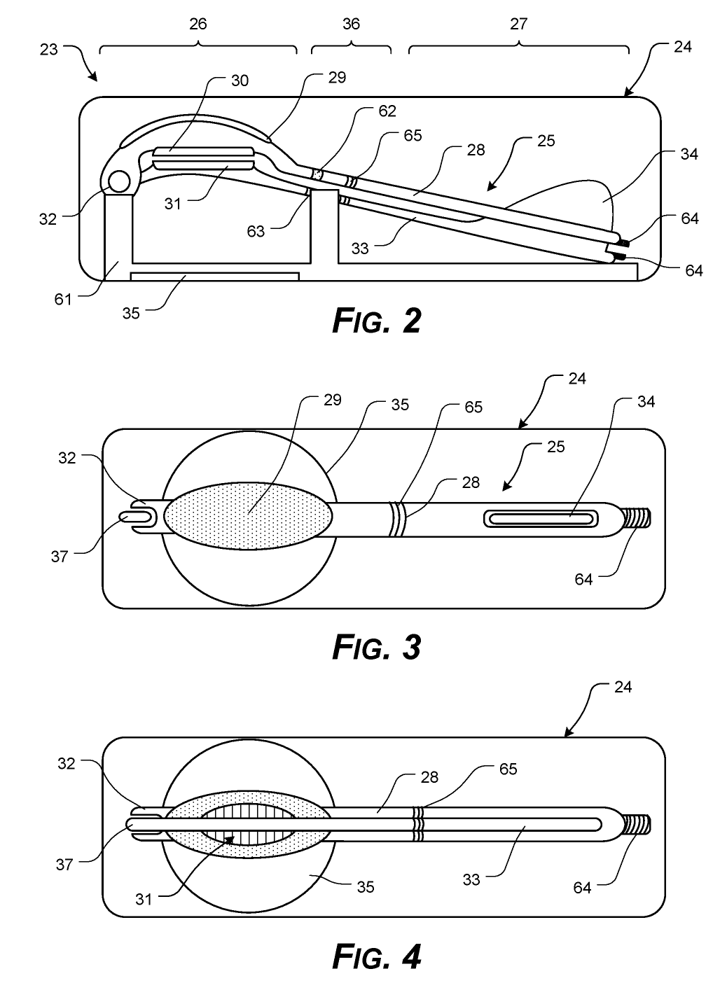

FIGS. 2-4 illustrate an exemplary embodiment of the transplant system 23, according to the present disclosure. As illustrated, the transplant system 23 comprises a container vessel 24 with a support tray 61 disposed within the interior chamber or cavity of the container vessel 24. An exemplary transplant device 25 is positioned atop or at least partially within the support tray 61.

In various exemplary embodiments, the container vessel 24 may optionally contain a viewing port 35 to visualize transplant tissue 80 mounted on the transplant tissue container vessel 25. The transplant device 25 may optionally be secured in the container vessel 24 by a variety of mechanisms. In certain exemplary embodiments, the container vessel 24 may optionally be secured to the transplant device 25 by interaction of a externally threaded portion 65 formed on the first arm 28 and the second arm 33 and a coupling sleeve 76, as illustrated in FIG. 30.

FIG. 3 illustrates a top view of the transplant system 23 further illustrating the container vessel 24 and transplant device 25. As illustrated, the tissue support element 29 of the first arm 28 in the distal portion 26 may optionally be formed so as to have a substantially oval shape. Although other embodiments may take different geometrical shapes, the tissue support element 29 is formed so as to further facilitate proper handling of the transplant tissue 80 during placement as it mimics a natural curvature of the cornea. Also visible from the top perspective is the pivot element 32 of the first arm 28 and second arm 33 in the distal portion 26. This embodiment represents the articulation area 37 in the distal portion 26. In certain exemplary, nonlimiting embodiments, the container vessel 24, or at least the distal portion 26 of the container vessel 24, can be seen through at least a portion of the viewing port 35.

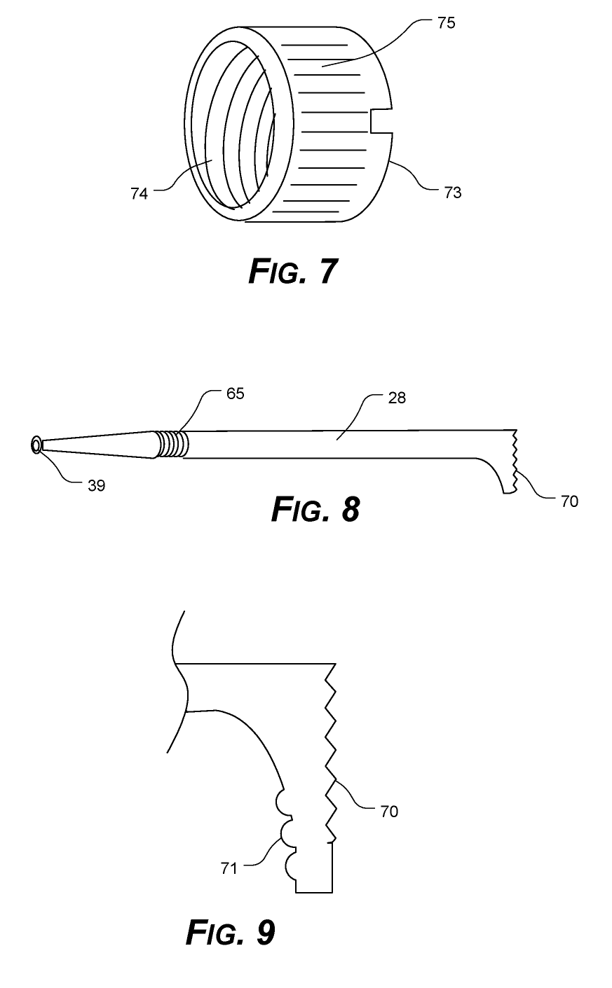

In certain exemplary, nonlimiting embodiments, the proximal portion 27 may optionally include a lockable arrangement 64, as illustrated in FIGS. 7-9, that can immobilize or lock the first arm 28 and the second arm 33 in the closed (or another determined) position. In various exemplary, nonlimiting embodiments, the lockable arrangement 64 comprises mating portions of an externally threaded protrusion or a clamp and can prevent the inadvertent release of transplant tissue 80. By unlocking the lockable arrangement 64, the first arm 28 will no longer be secured relative to the second arm 33, thereby enabling the user to rotate the first securing pad 30 relative to the second securing pad 31 in the distal portion 26 to release the secured transplant tissue 80. The opposing peripheral portions of the secured transplant tissue 80 are freed upon actuation, allowing the transplant tissue 80 in a draped configuration to be released from the transplant device 25. Once released, the transplant tissue 80 naturally unfurls from the draped configuration into the shape of the eye, as well be appreciated by those of ordinary skill in the art.

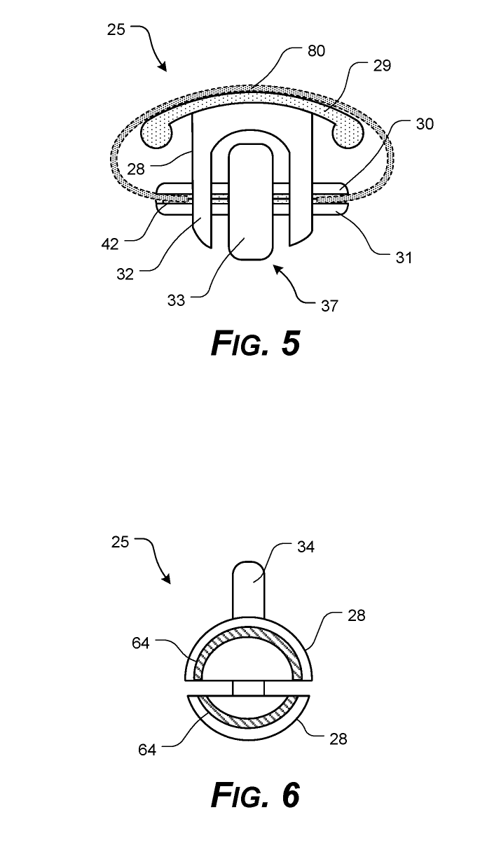

FIG. 4 illustrates a bottom view of the transplant system 23 illustrating the container vessel 24 and transplant device 25. As illustrated, the second arm 33 articulates, relative to the first arm 28, via the pivot element 32. The container vessel 24 may optionally include the viewing port 35. In certain exemplary, nonlimiting embodiments, the proximal portion 27 may optionally also have a lockable arrangement 64 that may optionally lock the first arm 28 to the second arm 33, and a closed position. In certain exemplary embodiments, the container vessel 24 may optionally be secured to the transplant device 25 by interaction of the externally threaded portion 65 on the first arm 28 and the second arm 33 and a coupling sleeve 76.

FIG. 5 shows exemplary transplant tissue 80 secured in a draped configuration atop at least a portion of the tissue support element 29. In FIG. 5, the front view of the transplant device 25 details the articulation area 37 where the first arm 28 articulates with respect to the second arm 33, via pivot element 32. The front profile of the transplant tissue securing mechanism that secures the transplant tissue 80 in place is illustrated in situ with the first securing pad 30 and second securing pad 31 attached or coupled to the first arm 28 and the second arm 33, respectively. The front profile detail of the tissue support element 29 of the transplant device 25 illustrates the side-to-side curvature of the tissue support element 29. In various exemplary embodiments, the edge portions of the tissue support element 29 may optionally also have overhung edges to reduce damage to the transplant tissue 80 during handling. Certain exemplary embodiments may optionally also contain a securing element 42 to assist in gentle release of secured transplant tissue 80. As illustrated in more detail, this figure is one example of the transplant tissue 80 location within the transplant device 25 and in a draped configuration.

In FIG. 6, the rear view of the transplant device 25 proximal portion 27 details the layout of one possible embodiment of the release element 34. As illustrated, the release element 34 is depicted as a fin like projection that arises from the second arm 33 and emerges through an aperture formed in the first arm 28 of the transplant device 25. In certain exemplary, nonlimiting embodiments, the proximal portion 27 may optionally also have a lockable arrangement 64 that may optionally lock the first arm 28 to the second arm 33.

FIG. 7 illustrates a front-side view of an internally threaded locking cap 73 of an exemplary, nonlimiting embodiment of a locking mechanism. The first arm 28 may optionally attach to the second arm 33 in the proximal portion 27 utilizing lockable arrangement 64 (as illustrated in FIG. 6). The internally threaded screw pattern 74 couples with the transplant device 25 at lockable arrangement 64. In various exemplary embodiments, the internally threaded locking cap 73 comprises an external gripping surface 75.

FIG. 8 illustrates a side view of an exemplary embodiment of the first arm 28. The proximal end of the first arm 28 comprises an elongated end that contains gripping portion 70 that is used to release first arm 28 from second arm 33.

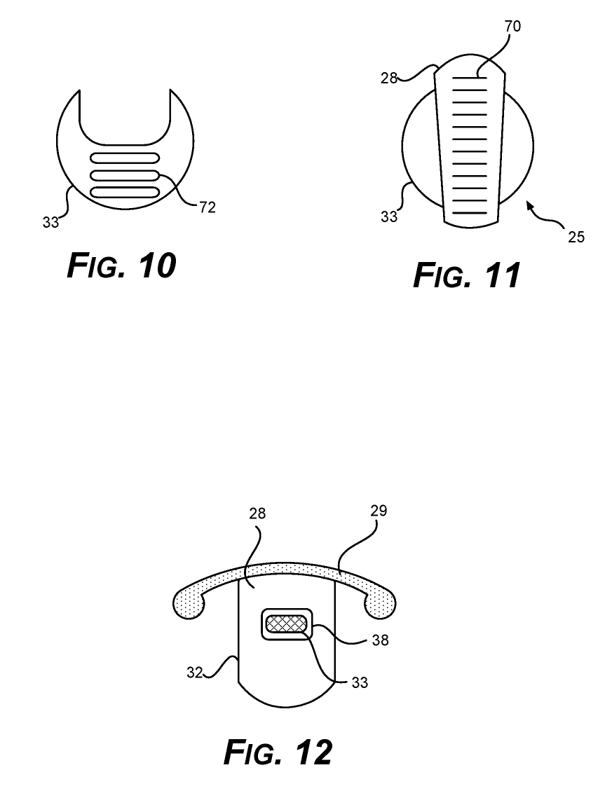

FIG. 9 illustrates a more detailed side view of an exemplary, nonlimiting embodiment of a locking mechanism created by the proximal portion 27 of first arm 28. One or more semicircular catches 71 are formed behind the gripping portion 70 on the elongated end of the first arm 28. In various exemplary embodiments, the semicircular catches 71 latch on to the second arm 33 of the transplant device 25.

FIG. 10 illustrates a rear view of an exemplary, nonlimiting embodiment of a second arm 33 locking mechanism. In this view, the second arm 33 is in the unlatched position and depicts exemplary locking grooves 72 that are formed so as to receive the semicircular catches 71 (as illustrated in FIG. 9).

FIG. 11 illustrates a rear view of an exemplary, nonlimiting embodiment of the locking mechanism wherein the first arm 28 is latched to the second arm 33. Also illustrated in this view is the gripping portion 70 of the elongated proximal end of the first arm 28.

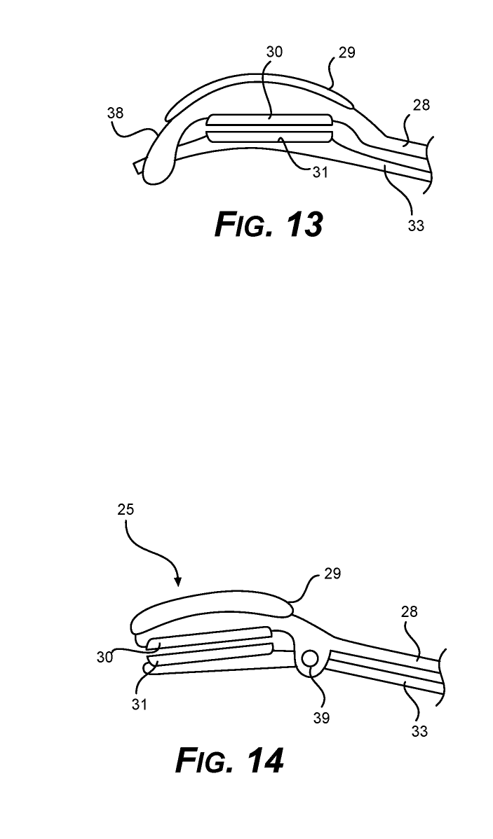

FIGS. 12-13 illustrate an exemplary embodiment of the transplant device 25 with removable shafts. In FIG. 12, a portion of the second arm 33 emerges through an aperture or opening 38 in the first arm 28. This is one example of how the first arm 28 and the second arm 33 may optionally function to secure the transplant tissue 80 (not illustrated in FIG. 12) in place. A side view of the removable shaft configuration of the transplant device 25 is illustrated in FIG. 13. As illustrated, the second arm 33 protrudes through the first arm 28 at the aperture or opening 38 in the first arm 28. The relationship of the tissue support element 29 as well as the first securing pad 30 and the second securing pad 31 are also illustrated.

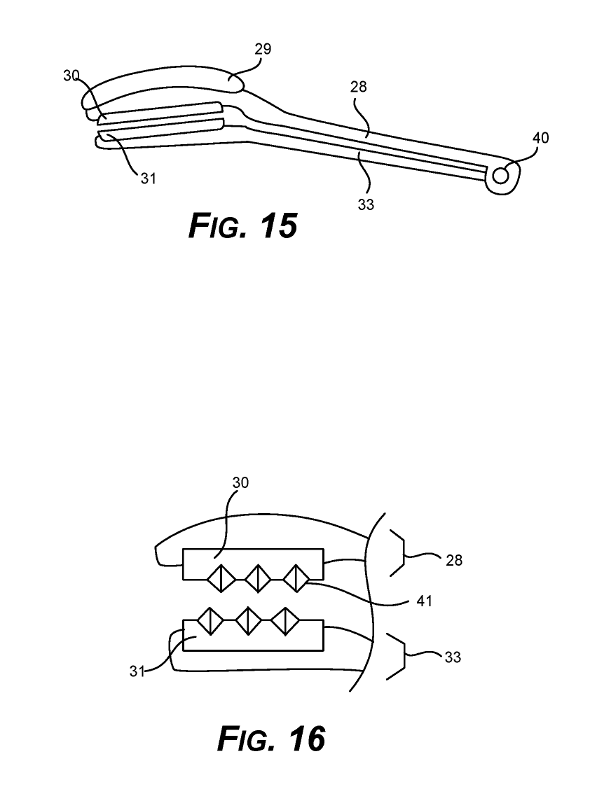

FIGS. 14-15 illustrate alternate articulation embodiments for the transplant device 25. As illustrated in FIG. 14, the transplant device 25 includes a centrally located pivot element 39. As shown, the articulation apparatus for pivot element 39 emerges from first arm 28 of the transplant device 25 and is secured to the second arm 33. This configuration may optionally be reversed in other examples such that the pivot element 39 may optionally emerge from the second arm 33 and be secured on the first arm 28. Depicted here in relation to the pivot element 39 are the tissue support element 29 of the first arm 28 as well as the first securing pad 30 and the second securing pad 31. Another embodiment of the pivot element 32, for example in the proximal portion 27 of the transplant device 25 is depicted in FIG. 15. This proximal pivot element 40 allows either the first arm 28 or the second arm 33 of the transplant device 25 to move freely. In this example the articulation apparatus for pivot element 40 emerge from the first arm 28 and secured on the second arm 33 of the transplant device 25. Again this is one example but the articulation apparatus configuration may optionally be reversed such that the articulation apparatus could emerge from the second arm 33 and is then secured on the first arm 28 of the transplant device 25. Depicted here in relation to the pivot element 40 are the tissue support element 29 of the first arm 28 as well as the first securing pad 30 and the second securing pad 31.

As illustrated in greater detail in FIG. 16, in certain exemplary embodiments of the transplant tissue securing mechanism, the first securing pad 30 and the second securing pad 31, which receive at least portions of the transplant tissue 80 may optionally be located on either the first arm 28, the second arm 33, or both. In various exemplary embodiments, the first securing pad 30 and the second securing pad 31 may have one or more protrusions on a transplant tissue 80 contacting surface. The protrusions may be configured to extending into the transplant tissue 80 and thereby more firmly secure the transplant tissue 80 in the transplant tissue securing mechanism. For example, the transplant tissue securing mechanism may optionally comprise of pyramidal 41 or rounded spires 66 that clasp the transplant tissue 80 (not illustrated in FIG. 16) for locking. The number of spires or other protrusions and their arrangement on a securing pad (first securing pad 30 and/or second securing pad 31) can vary.

With spires located on either or both first securing pad 30 and the second securing pad 31. Thus, in certain exemplary, nonlimiting embodiments a tissue support element 29 may include a transplant tissue securing mechanism having opposing first securing pad 30 and the second securing pad 31 to clasp or clamp a portion of the transplant tissue 80. In certain exemplary, nonlimiting embodiments, the tissue support element 29 may include both an upper surface with a tissue support element 29 for contacting a middle or central portion of the transplant tissue 80 and a transplant tissue securing mechanism with opposing first securing pad 30 and second securing pad 31 to clasp or clamp a portion of the transplant tissue 80, such as opposing peripheral portions of the transplant tissue 80. This mounting configuration enables the transplant tissue 80 to be releasably secured in a draped configuration. As should be appreciated by those skilled in the art, a draped configuration advantageously allows for convenient handling and accurate placement of the transplant tissue 80.

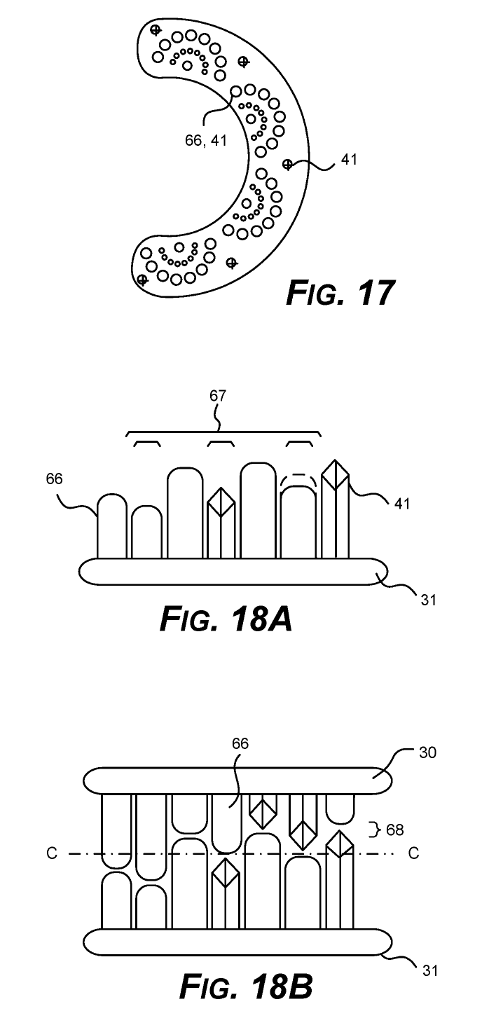

FIG. 17 illustrates a top view of an exemplary, nonlimiting embodiment of a securing pad. The illustrated securing pad may optionally either be the first securing pad 30 and/or the second securing pad 31. The securing pad (first securing pad 30 and/or second securing pad 31) may optionally comprise a combination of rounded spires 66 or pyramidal spires 41. The rounded spires 66 and/or pyramidal spires 41 may optionally be arranged in a semicircular pattern, as illustrated, or in a random or other predetermined fashion.

In various exemplary, nonlimiting embodiments, opposing surfaces of the first securing pad 30 and the second securing pad 31 are substantially planar. Alternatively, opposing surfaces of the first securing pad 30 and the second securing pad 31 follow the curvature of the pre-cut transplant tissue 80. Opposing surfaces of the first securing pad 30 and the second securing pad 31 act to secure the transplant tissue 80 along at least a rim portion of the transplant tissue 80. In various exemplary embodiments, the transplant tissue 80 the opposing, contacting surfaces of the first securing pad 30 and the second securing pad 31 may have a horseshoe shape, as illustrated in FIG. 17, to contact and secure outer peripheral portions of the transplant tissue 80.

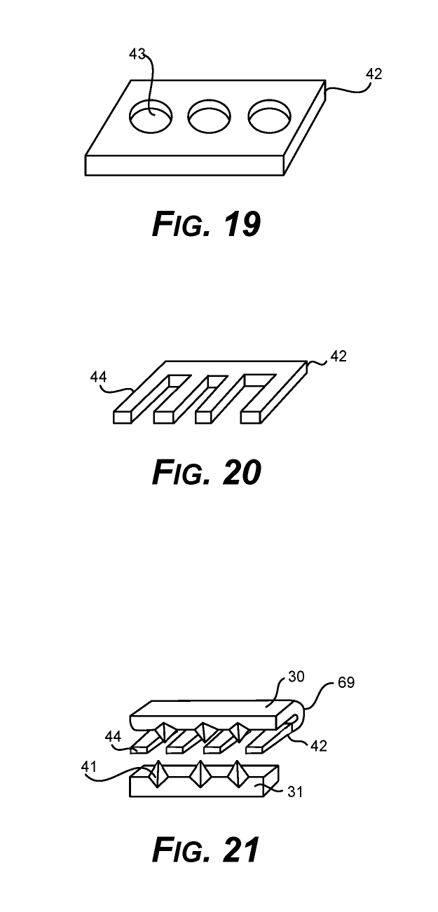

FIG. 18A illustrates a side view of an exemplary, nonlimiting embodiment of a transplant tissue securing pad (first securing pad 30 and/or second securing pad 31). This embodiment illustrates an optional spire arrangement 67, comprising rounded spires 66, and pyramidal spires 41. The spire arrangement 67 may optionally also vary in height and placement. FIG. 18B illustrates an optional arrangement of the first securing pad 30 and second securing pad 31. In this arrangement, at least some of the rounded spires 66 on one securing pad (first securing pad 30 and/or second securing pad 31) conjugate with at least some of the pyramidal spires 41 on the corresponding securing pad (first securing pad 30 and/or second securing pad 31). In spire arrangement 67, a determined gap 68 is formed between opposing rounded spires 66 and/or pyramidal spires 41 located towards a front of the securing pad (first securing pad 30 and/or second securing pad 31), when the transplant device 25 is in the closed position. The gap 68 is greater than any optional gap between opposing rounded spires 66 and/or pyramidal spires 41 located in other portions of the securing pad (first securing pad 30 and/or second securing pad 31), when the transplant device 25 is in the closed position. The gap 68, if included, acts to further stabilize the transplant tissue 80, when the transplant tissue 80 is secured between the first securing pad 30 and the second securing pad 31. In various exemplary embodiments, the distance created by the gap 68 decreases as the pattern of the spire arrangement 67 continues towards a back of the securing pad (first securing pad 30 and/or second securing pad 31). In this arrangement, the heights of the rounded spires 66 and the pyramidal spires 41 vary from the front to the back of the securing pad (first securing pad 30 and/or second securing pad 31) as further illustrated by the centerline, C.

FIGS. 19-21 illustrate various exemplary embodiments of components or elements that may be incorporated into portions of the first securing pad 30 and/or second securing pad 31 to facilitate securing and/or release of the transplant tissue 80 from the first securing pad 30 and/or the second securing pad 31. In one exemplary embodiment, as illustrated in FIG. 19, at least one of the first securing pad 30 and/or the second securing pad 31 optionally comprise a securing element 42 with apertures or perforations 43 and may optionally be of various thicknesses. The securing element 42 may optionally be located either on the first arm 28 (not illustrated in FIG. 19), the second arm 33 (not illustrated in FIG. 19), or both the first arm 28 and the second arm 33 and may optionally be co-located with the first securing pad 30 and the second securing pad 31 on the transplant device 25 (not illustrated in FIG. 19).

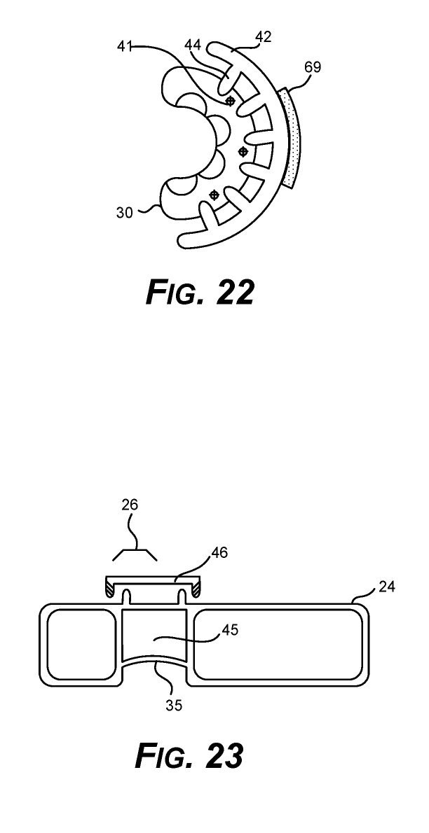

An alternate embodiment of the securing element 42 is illustrated in FIG. 20. In this exemplary embodiment, the securing element 42 comprises one or more comb like elongations 44 that may optionally locate between the spires of the first securing pad 30 and the second securing pad 31. Again, the securing element 42 may optionally be located either on the first arm 28 (not illustrated in FIG. 20), the second arm 33 (not illustrated in FIG. 20), or both of the first arm 28 and the second arm 33 and may optionally be co-located with the first securing pad 30 and the second securing pad 31 on the transplant device 25 (not illustrated in FIG. 20). The interaction of the first securing pad 30 and the second securing pad 31 and the securing element 42 is shown in FIG. 21. In this illustration, the securing element 42 can be seen positioned between the pyramidal spires 41 of either the first securing pad 30 or the second securing pad 31. The securing element 42 may optionally attach to either the first arm 28 or the second arm 33 of transplant device 25 at or proximate position 69.

FIG. 22 illustrates a top view of an exemplary embodiment of the securing element 42. The first securing pad 30 is illustrated for reference. The securing element 42 is located between the first securing pad 30 and the second securing pad 31. This embodiment may optionally also be comprised of comb like elongations 44 that may be optionally located between the spires of the first securing pad 30 and the second securing pad 31. In this illustration, the comb like elongations 44 are located proximate the pyramidal spires 41, towards the back of the second securing pad 31. The location of securing element 42 towards the back of the first securing pad 30 and the second securing pad 31 can facilitate easier release of the transplant tissue 80, as this area can contain greater amounts of pyramidal spires 41 for improved purchase or grasping power.

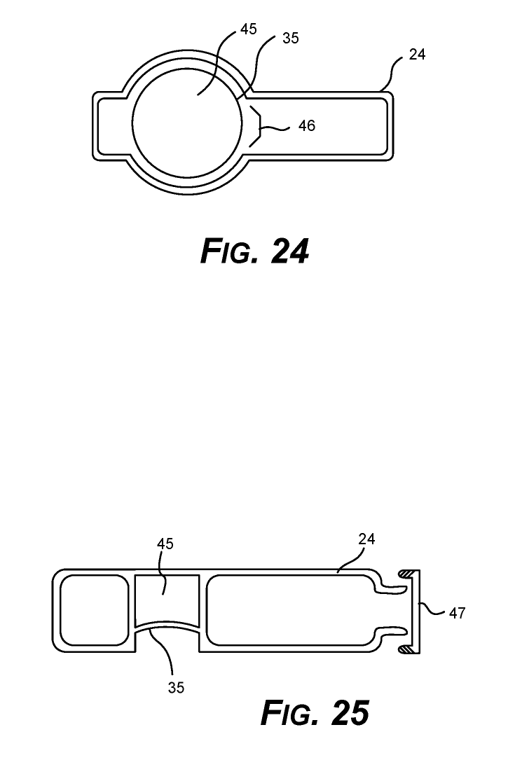

FIGS. 23-29 illustrate various exemplary embodiments of the container vessel 24. In FIG. 23, the container vessel 24 is shown in more detail in a side cross-sectional view. This example depicts a top access area 46 into the container vessel 24. The top access area 46 is contiguous with a transplant tissue cell or cavity 45 and a transplant tissue viewing port 35. The transplant device 25 may optionally be introduced into the container vessel 24 using this example of a top access area 46. The transplant tissue cell or cavity 45 defines an area where the distal portion 26 of the transplant device 25 is secured during transport. Once placed into the container vessel 24, the transplant tissue 80 secured on the transplant device 25 may optionally be evaluated using the viewing port 35. This is one example of the placement of these components as their arrangement can vary. A top view of the transplant device 25 configuration mentioned in FIG. 23 is illustrated in FIG. 24. The top access area 46 is contiguous with the transplant tissue cell or cavity 45 and also with the viewing port 35.

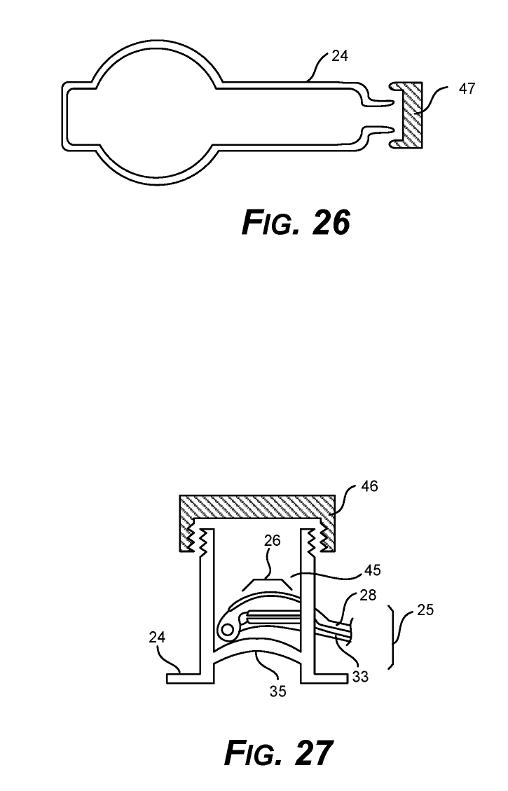

An exemplary embodiment of the container vessel 24 is shown in FIG. 25 with a side access area 47. As with the example of the proposed instrument in FIG. 23, the side access area 47 will also enable the transplant device 25 to be loaded and secured into the container vessel 24 during storage and transport conditions. The side access area 47 configuration may optionally also enable the distal portion 26 to be placed in the transplant tissue cell or cavity 45, which can then be evaluated using the viewing port 35. A top view of the transplant device 25 configuration mentioned in FIG. 25 is illustrated in FIG. 26. Although they could be visible in the actual device, the transplant tissue cell or cavity 45 and viewing port 35 are not illustrated in FIG. 26. As with the illustration in FIG. 25, the container vessel 24 communicates with the side access area 47 point in a similar fashion as depicted in FIG. 25.

FIG. 27 illustrates an exemplary embodiment of the transplant tissue cell or cavity 45 of the container vessel 24. This detailed view demonstrates one example of the transplant device 25 fitment inside the transplant tissue cell or cavity 45. The distal portion 26 of the transplant device 25, as depicted in FIG. 2, is locked or secured into place inside the transplant tissue cell or cavity 45 with the first arm 28 and the second arm 33 oriented top and bottom respectively. Orientation of the transplant device 25 inside the transplant tissue cell or cavity 45 is not limited to that as illustrated in FIG. 27 and can vary significantly. It can be possible to evaluate the transplant tissue 80 utilizing the viewing port 35. In this exemplary embodiment, the transplant tissue cell or cavity 45 apparatus is optionally configured to include top access area 46.

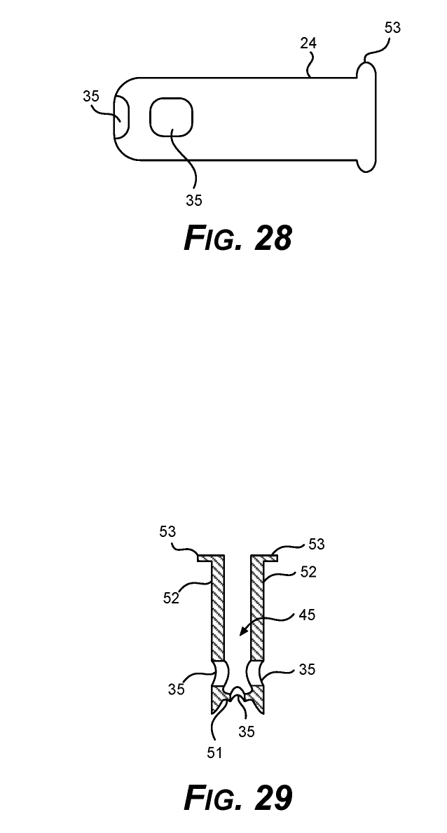

In FIG. 28, a side view of an exemplary embodiment of transplant tissue container vessel 24 is illustrated. This embodiment is a compact version of the transplant tissue container vessel 24 with flanges 53 on the outer rim to couple with the transplant device 25. The closed end of container vessel 24 may optionally include one or more viewing ports 35 comprised of convergent lenses for better transplant tissue 80 visualization. The viewing ports 35 may optionally be located at random areas proximate a closed end of the container vessel 24.

In certain exemplary embodiments, the closed end of the container vessel 24 may optionally have a bottom wall 51 illustrated as a cross-sectional view in FIG. 29. The bottom wall 51 may optionally absorb mechanical shocks exerted on the transplant tissue 80 during transport. Another embodiment may optionally have a band containing viewing ports 35 that span the circumference of the container vessel 24. In certain exemplary embodiments, the bottom wall 51 and sidewalls 52 may optionally include viewing ports 35 located in multiple locations along bottom wall 51 and sidewalls 52. The bottom wall 51 and sidewalls 52 may optionally also include locking mechanisms for the transplant device 25 (not illustrated in FIG. 29). The open end of the container vessel 24 may optionally also have flanges 53 on the outer rim to couple with the transplant device 25. Certain exemplary embodiments of the apparatus may optionally have sidewalls 52 and a bottom wall 51 in a cylindrical shape or of various shapes, the thickness and the length of the sidewalls 52 and bottom wall 51 can vary.

FIG. 30 is cutaway front-side view of an exemplary, nonlimiting embodiment of a coupling sleeve 76 that couples container vessel 24 to the transplant device 25. A grove 78 on the coupling sleeve 76 may optionally receive the flange 53 of the container vessel 24. Internally threaded portion, including internal threads 79 on the coupling sleeve 76 may optionally attach to externally threaded portion 65 on the first arm 28 and the second arm 33 of the transplant device 25. The outer surface of the coupling sleeve 76 may optionally contain gripping surface 77, which may optionally be comprised of various patterns that enhance grip.

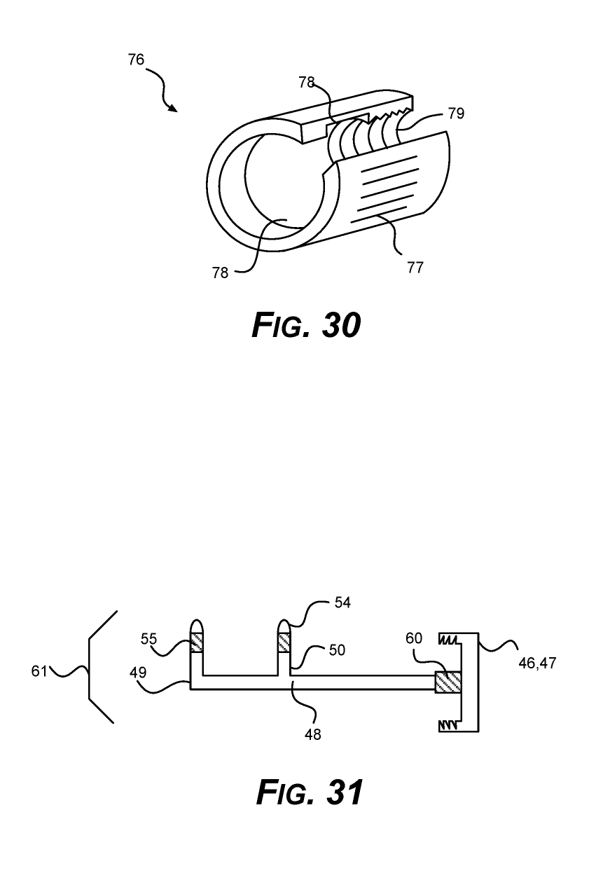

FIG. 31 illustrates an example of a restraining mechanism, for example a support tray 61, for the transplant device 25 inside the container vessel 24. In certain exemplary embodiments, the support tray 61 may optionally consist of a trunk 48 and with a protruding distal branch 49 and protruding proximal branch 50. The trunk 48 of the support tray 61 may optionally be attached to the top access area 46 or side access area 47 using a removable mechanism 60, for example a luer type lock, or may optionally be permanently attached. In various exemplary embodiments, the luer type lock comprises of two different inner diameters and fitment at each end. The inner surface of the proximal end of the luer fitting is tapped to receive the distal end of the transplant device, while the outer surface of the proximal end of the luer fitting comprises of patterned grooves to enhance grip. The distal end of the luer fitting is moveable and is held in place by the lip of the container vessel 24. The luer fitting facilitates docking of the container vessel 24 and the transplant device 25 and provides an airtight fit.

The protruding distal branch 49 and protruding proximal branch 50 of the support tray 61 may optionally be configured to receive, secure, and orient the transplant device 25 at orientation points or grooves 54 and 55.

FIG. 32 illustrates a front-side view of the support tray 61 showing the details of the orientation points or grooves 54 and 55. The orientation points or grooves 54 and 55 ensure proper orientation of the transplant device 25 so as to not compromise the integrity of the transplant tissue 80 during transport. In certain exemplary embodiments of support tray 61, the orientation points or grooves 54 and 55 located on protruding distal branch 49 and protruding proximal branch 50 may optionally be of similar shapes, sizes, or materials. In this example, the orientation point or groove 55 located on the protruding distal branch 49 is square shape and is vastly different from orientation point or groove 54 located on protruding proximal branch 50. Utilization of different shapes on either protruding distal branch 49 and protruding proximal branch 50 of the support tray 61 may optionally also help ensure proper alignment of the transplant device 25.

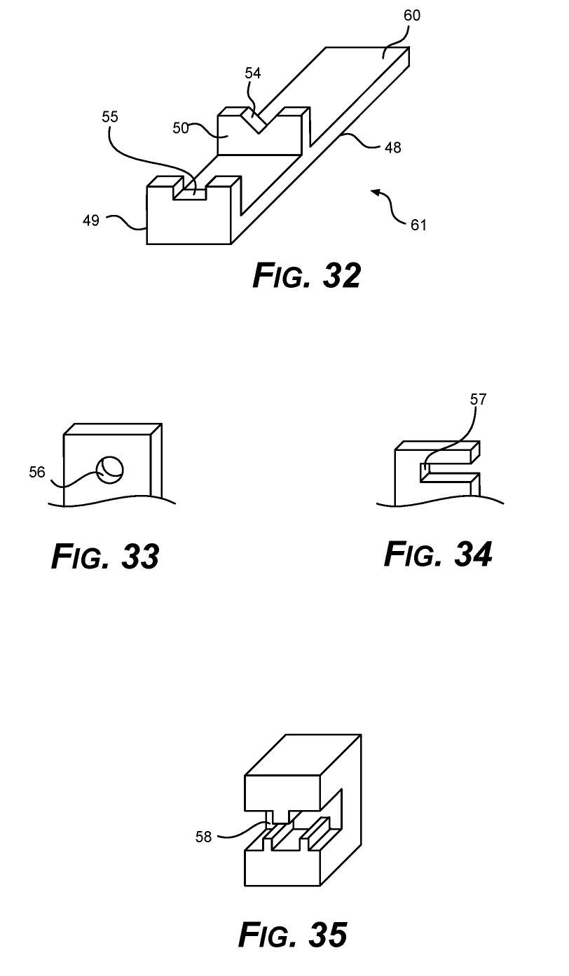

Other embodiments of orientation grooves are illustrated in FIGS. 33, 34 and 35. In one example the orientation groove may optionally have a rounded shape 56 or have a rectangular shape 57. These are only a few examples of the possible shape, size and location of the orientation grooves, it should be noted that many variations are possible. Additional components that may optionally assist in securing and restraining the transplant device 25 are illustrated in FIG. 35. In this exemplary embodiment, a rectangular groove 57, such as that illustrated in FIGS. 33, 34, may optionally be further configured with flanges 58 that may optionally be disposed in a fashion to receive the transplant device 25. While this example depicts the shape of the orientation pin to be rectangular 57, other shapes and sizes are possible with mechanisms similar to the flanges 58 utilized as appropriate.

The transplant system 23 may optionally be used to store, transport, and place a transplant tissue 80, which may optionally vary in size and shape according to the surgeons specifications. For example, most transplant tissues 80 may optionally be disk shaped with diameters ranging from 5 mm to 9 mm. In some instances, the cornea transplant tissues 80 may be of irregular shape, such as elliptical, oval, square, rectangular, etc.

Advantageously, embodiments of the transplant device 25 may be configured to hold the transplant tissue 80 in a draped configuration.

As discussed herein, the container vessel 24 may optionally be used to transport a donor transplant tissue 80 secured in the transplant device 25, with or without the support tray 61, to the surgical center. The transplant device 25 may optionally be used to place the donor transplant tissue 80 in a specific transplant site within the anterior chamber of a recipient's eye. The container vessel 24, the transplant device 25, or support tray 61 may optionally be provided to a transplant tissue 80 processing facility, such as an eye bank, that prepares the transplant tissue 80 allografts and transports them to a surgical facility for transplantation. The transplant tissue container vessel 24 and the transplant device 25 or support tray 61 may optionally be provided separately or in a kit.

A transplant tissue 80 processing entity, such as a transplant tissue bank or an eye bank, can prepare the transplant tissue 80 for transplant by cutting it to the surgeon's specifications and ensuring the transplant tissue 80 is of appropriate quality. For example, to prepare transplant tissue 80 for transplant, donor transplant tissue 80 is harvested with scleral rims and a quality assessment is made by counting the endothelial cells and assessing the corneal thickness. With the endothelial side facing up, the desired corneal endothelial plug of the desired size is then punched in this posterior aspect. This is one example of transplant tissue 80 preparation at a transplant tissue bank. The transplant tissue 80 is mounted on the transplant device 25. Additional quality assessments, which can include endothelial cell counts and pachymetry (corneal thickness assessment), which are only possible with the transplant device 25 described herein, may optionally be made before transport to the surgeon.

More specifically, the technician, after creating a sterile field, unpackages the transplant tissue container vessel 24, transplant device 25, or support tray 61 and places them in the sterile field. After punching the corneal endothelial surface with the appropriate size punch, the technician decouples the transplant tissue 80 at the level of Descement's membrane leaving a few adhesion areas alongside the sagittal plane. The transplant device 25 is aligned along the sagittal plane of the donor cornea and oriented such that the first securing pad 30 on the first arm 28 faces the technician, as illustrated in FIG. 4. Using a small forceps or similar medical tool, the technician grabs one end of the rounded edge of the transplant tissue 80 and places on to the pyramidal spires 41 of the first securing pad 30 on the first arm 28. The same procedure is repeated to the other end of the transplant tissue 80.

After aligning the two ends of the transplant tissue 80 on to the first securing pad 30, the second arm 33 of the transplant device 25 may optionally be introduced to secure the transplant tissue 80 in place, via interaction of the first securing pad 30 and the second securing pad 31. In other embodiments, the first arm 28 may optionally be locked to the second arm 33 utilizing the lockable arrangement 64, semicircular catches 71, and/or locking grooves 72.

Once mounted on the transplant device 25, the remainder of the endothelial adhesions may optionally be uncoupled from the Descement's membrane so that the endothelium from the transplant tissue 80 is completely free. The transplant device 25 along with the secured transplant tissue 80 may optionally now be either coupled with the support tray 61 or placed directly inside the container vessel 24 for further evaluation utilizing the viewing port 35 and eventual transport.

Prior to the introduction of the transplant device 25 and transplant tissue 80, the transplant tissue cell or cavity 45 of the container vessel 24 is rinsed with the transport medium such as OPTISOL GS® (Bausch and Lomb, NY) to lubricate and mitigate bubble formation. The transplant device 25 could be placed in the orientation point or groove 54 and orientation point or groove 55 of the support tray 61 and then introduced inside the container vessel 24 in a specific orientation. Alternatively, the transplant device 25 may optionally be directly placed inside the container vessel 24 using various described embodiments. One example of the final assembly is the transplant tissue cell or cavity 45 filled with transport medium, for example OPTISOL GS, after which the cap assembly either top access area 46 or side access area 47 is placed and locked. Once this system is assembled, the transplant tissue 80 may optionally be assessed utilizing the viewing port 35 of the container vessel 24. The transplant system in FIG. 1 with the transplant tissue 80 may optionally be stored for up to 72 hours or transported immediately to the surgeon.

At the surgical site, the transplant system 23 is removed from its packaging and placed in a sterile area. The surgeon inspects the transplant tissue 80 and verifies transplant tissue 80 suitability. The surgeon prepares the transplant tissue 80 recipient for endothelial keratoplasty according to accepted protocols. A lateral incision is made on the lateral aspect of the recipient's cornea at the level of the sclero-corneal junction. In certain exemplary, nonlimiting embodiments, the lateral incision may optionally measure between 3 mm to 8 mm in length.

The surgeon then un-screws the locking screw, either top access area 46, side access area 47 or coupling sleeve 76, removes the transplant device 25 from the container vessel 24 and support tray 61 if coupled, and can grasp the transplant device 25 in the proximal portion 27. In certain exemplary embodiments, the internally threaded locking cap 73 may optionally be uncoupled from lockable arrangement 64 to release the locked first arm 28 and the second arm 33 of the transplant device 25. The distal portion 26 of the transplant device 25 may optionally be inserted into the recipient's anterior chamber, via the lateral incision. The distal portion 26 is guided to the appropriate surgical location and placed. The tissue support element 29 of the distal portion 26 of the transplant device 25 can be used to temporarily hold the transplant tissue 80 at the transplant site while the transplant tissue 80 is released from the transplant device 25.

In certain exemplary embodiments, the transplant tissue 80 is released by applying pressure on the “shark fin” release element 34 located on the proximal portion 27 of the transplant device 25. In certain exemplary embodiments, the transplant tissue 80 can be released by applying pressure to gripping portion 70 located on the elongated end of the first arm 28 at the proximal portion 27 of the transplant device 25. Applying pressure will unlatch mechanisms semicircular catches 71 and locking grooves 72 to release the transplant tissue 80 on the distal end of the transplant device 25. The surgeon can continue to hold the transplant tissue 80 in place with the tissue support element 29 of the transplant device 25 until a satisfactory adhesion of the transplant tissue 80 has been achieved.

The various embodiments of the components of the transplant system 23, the container vessel 24, the transplant devices 25, and the trunk 48 may optionally be constructed of materials that are commonly used in similar devices that could vary from stainless steels, acrylic polymers or other polymer materials. The materials such as stainless steel, titanium, titanium alloy, surgical steel, metal alloys, various polymers, various flexible materials, various rubber materials, or combinations of various materials thereof need to be biocompatible and therefore may optionally be suitable in the construction of the different embodiments of the components of the transplant system 23, the container vessel 24, the transplant devices 25, or the trunk 48 disclosed herein.

While the presently disclosed systems, methods, and/or apparatuses have been described in conjunction with the exemplary embodiments outlined above, the foregoing description of exemplary embodiments of the presently disclosed systems, methods, and/or apparatuses, as set forth above, are intended to be illustrative, not limiting and the fundamental disclosed systems, methods, and/or apparatuses should not be considered to be necessarily so constrained. It is evident that the presently disclosed systems, methods, and/or apparatuses is not limited to the particular variation set forth and many alternatives, adaptations modifications, and/or variations will be apparent to those skilled in the art.

Where methods and steps described above indicate certain events occurring in certain order, those of ordinary skill in the art having the benefit of this disclosure would recognize that the ordering of certain steps may be modified and that such modifications are in accordance with the variations of the present disclosure. Additionally, certain of the steps may be performed concurrently in a parallel process when possible, as well as performed sequentially as described above.

Furthermore, where a range of values is provided, it is understood that every intervening value, between the upper and lower limit of that range and any other stated or intervening value in that stated range is encompassed within the presently disclosed systems, methods, and/or apparatuses. The upper and lower limits of these smaller ranges may independently be included in the smaller ranges and is also encompassed within the presently disclosed systems, methods, and/or apparatuses, subject to any specifically excluded limit in the stated range. Where the stated range includes one or both of the limits, ranges excluding either or both of those included limits are also included in the presently disclosed systems, methods, and/or apparatuses.

It is to be understood that the phraseology of terminology employed herein is for the purpose of description and not of limitation. Unless defined otherwise, all technical and scientific terms used herein have the same meaning as commonly understood by one of ordinary skill in the art to which the presently disclosed systems, methods, and/or apparatuses belongs.

In addition, it is contemplated that any optional feature of the inventive variations described herein may be set forth and claimed independently, or in combination with any one or more of the features described herein.

Accordingly, the foregoing description of exemplary embodiments will reveal the general nature of the presently disclosed systems, methods, and/or apparatuses, such that others may, by applying current knowledge, change, vary, modify, and/or adapt these exemplary, non-limiting embodiments for various applications without departing from the spirit and scope of the presently disclosed systems, methods, and/or apparatuses and elements or methods similar or equivalent to those described herein can be used in practicing the presently disclosed systems, methods, and/or apparatuses. Any and all such changes, variations, modifications, and/or adaptations should and are intended to be comprehended within the meaning and range of equivalents of the disclosed exemplary embodiments and may be substituted without departing from the true spirit and scope of the presently disclosed systems, methods, and/or apparatuses.

Also, it is noted that as used herein and in the appended claims, the singular forms “a”, “and”, “said”, and “the” include plural referents unless the context clearly dictates otherwise. Conversely, it is contemplated that the claims may be so-drafted to require singular elements or exclude any optional element indicated to be so here in the text or drawings. This statement is intended to serve as antecedent basis for use of such exclusive terminology as “solely”, “only”, and the like in connection with the recitation of claim elements or the use of a “negative” claim limitation(s).

What is claimed is:

1. A transplant tissue storage, transport, and/or placement apparatus, comprising:
a transplant device extending from a distal portion to a proximal portion;
a tissue support element formed in said distal portion, said tissue support element having an upper surface having a tissue support element configured to support a central portion of transplant tissue, and a transplant tissue securing mechanism with opposing first securing pad and said second securing pad configured for clasping opposing peripheral portions of transplant tissue in a draped configuration; and
a lockable arrangement formed in said proximal portion, said lockable arrangement configured to release a secured transplant tissue from said mounting surface, upon actuation.

2. The apparatus of claim 1, wherein said proximal portion comprises a first arm extending proximally from said mounting surface and a first opposing securing pad, and a second arm extending proximally from a second opposing securing pad, said first arm hingedly connected to said second arm.

3. The apparatus of claim 2, wherein said transplant tissue securing mechanism comprises a first securing pad extending from said first arm, and a second securing pad extending from said second arm.

4. The apparatus of claim 2, wherein said hinged connection is located on a side of said mounting surface opposite of said proximal portion.

5. The apparatus of claim 1, further comprising a transplant tissue container vessel with an interior chamber configured to retain a cornea allograft storage media and house at least one of said transplant device and a transplant tissue support tray.

6. The apparatus of claim 5 wherein said transplant tissue container vessel comprises a viewing lens configured to provide magnification of at least a portion of said inner chamber.

7. The apparatus of claim 6, wherein said container vessel receives said distal end of said transplant device and is fitted with a luer type lock.

8. The apparatus of claim 6, wherein said transplant device is configured to be removably inserted into said container vessel's inner chamber.

9. The apparatus of claim 5, wherein said transplant tissue support tray comprises a transplant tissue mounting and placement mount for removably securing said transplant device.

10. The apparatus of claim 1, further comprising a transplant tissue support tray with a transplant tissue mounting and placement mount for removably securing said transplant device.

11. The apparatus of claim 1, wherein said opposing first securing pad and said second securing pad comprise a first opposing securing pad and a second opposing securing pad, and at least one opposing securing pad comprises a plurality of protrusions on a transplant tissue contacting surface.

12. The apparatus of claim 1, further comprising a lip within said transplant tissue container vessel interior chamber configured to receive said transplant device and a lip on said proximal end to allow docking with said transplant device.

13. The apparatus of claim 1, wherein said mounting surface and said transplant tissue securing mechanism are configured to secure a transplant tissue to prevent movement and orient said transplant tissue with said endothelial side facing towards said mounting surface.

14. The apparatus of claim 1, wherein at least one securing pad comprises a plurality of protrusions on a transplant tissue contacting surface.

15. The apparatus of claim 1, wherein said first securing pad and said second securing pad each have a horseshoe-shaped transplant tissue contacting surface.

16. The apparatus of claim 1, wherein said upper surface of said mounting surface is smooth and dome-shaped to mimics said concavity of said transplant tissue in said draped configuration.

17. The apparatus of claim 1, wherein said opposing first securing pad and said second securing pad have a first side and a second side, said first side configured for clasping a first outer peripheral portion of said transplant tissue, and said second side configured for clasping a second outer peripheral portion of said transplant tissue.

18. The apparatus of claim 17, wherein said first outer peripheral portion of said transplant tissue is substantially opposite said second outer peripheral portion of said transplant tissue.

19. A transplant tissue storage, transport, and/or placement system, comprising:
a transplant tissue container vessel with an interior chamber configured to retain a cornea allograft storage media; and
a transplant device having a distal portion and a proximal portion, wherein:
said distal portion having a tissue support element with an upper surface having a tissue support element configured to support a central portion of transplant tissue, and a transplant tissue securing mechanism with opposing first securing pad and said second securing pad configured for clasping opposing peripheral portions of transplant tissue in a draped configuration; and
said proximal portion having an lockable arrangement configured to release a secured transplant tissue from said mounting surface upon actuation.

20. The transplant tissue storage, transport, and/or placement system of claim 19, further comprising a transplant tissue support tray with a transplant tissue mounting and placement mount for removably securing said transplant device, wherein said transplant tissue support tray comprises a distal groove and a medial groove, and said transplant devices includes a first mating region configured to removably mate with said distal groove and a second mating region configured to removably mate with said medial groove.
